(19) United States
(12) Patent Application Publication
(10) Pub. No.: US 2012/0310137 A1
(43) Pub. Date: Dec. 6, 2012

(54) EYE SHUNT WITH POROUS STRUCTURE

(76) Inventor: Thomas A. SILVESTRINI, Alamo, CA (US)

(21) Appl. No.: 13/487,007

(22) Filed: Jun. 1, 2012

Related U.S. Application Data
(60) Provisional application No. 61/492,506, filed on Jun. 2, 2011.

(51) Int. Cl. A61M 1/00

(52) U.S. Cl. 604/8

(57) ABSTRACT

Disclosed are devices and methods for treatment of eye disease such as glaucoma. An implant is placed in the eye wherein the implant provides a fluid pathway for the flow or drainage of aqueous humor from the anterior chamber to the supraciliary or the suprachoroidal space, or to any space in the eye where drainage to that location will lower the intraocular pressure. The implant may include an elongate compressible structure and may be implanted in the eye using a delivery system that folds and or compresses the implant to provide a smaller cross-sectional area to allow a more minimally-invasive procedure. The compressibility of the implant is provided by a porous structure that may be collapsed by compression and delivered through a tube-shaped introducer.

### CROSS-REFERENCE TO RELATED APPLICATION

This application claims the benefit of U.S. provisional application Ser. No. 61/492,506, filed Jun. 2, 2011, which is incorporated by reference.

### BACKGROUND

This disclosure relates generally to methods and devices for use in treating glaucoma. The mechanisms that cause glaucoma are not completely known. It is known that glaucoma results in abnormally high pressure in the eye, which leads to optic nerve damage. Over time, the increased pressure can cause damage to the optic nerve, which can lead to blindness. Treatment strategies have focused on keeping the intraocular pressure down in order to preserve as much vision as possible over the remainder of the patient's life.

Glaucoma treatment includes the use of drugs that lower intraocular pressure through various mechanisms. The glaucoma drug market is approximately a two billion dollar market. The large market is mostly due to the fact that there are not effective surgical alternatives that are long lasting and complication-free. Drug treatments need much improvement, as they can cause adverse side effects and often fail to adequately control intraocular pressure. Moreover, patients are often not compliant in following proper drug treatment regimens, resulting in a lack of compliance and further symptom progression.

One way to treat glaucoma is to surgically implant a drainage device in the eye. The drainage device functions to allow aqueous humor to drain from the anterior chamber and thereby reduce the intraocular pressure. The drainage device is usually implanted using an invasive surgical procedure. Pursuant to one such procedure, a flap is surgically cut in the sclera. The flap is folded back to form a small pocket and the drainage device is inserted into the eye through the flap. This procedure can be quite problematic as the implants are large and can result in various adverse events such as infections, erosions, and scarring, leading to the need to re-operate.

Current implanted devices and surgical procedures for treating glaucoma have disadvantages and only moderate success rates. The procedures can be very traumatic to the eye and often require highly specialized surgical skills to properly place the drainage device in a proper location. Devices that drain fluid from the anterior chamber to a subconjunctival bleb beneath a scleral flap are known to be prone to infection and to occlude and cease working. In view of the foregoing, there is a need for improved devices and methods for the treatment of glaucoma.

### SUMMARY

An ocular implant is disclosed. The ocular implant includes an elongate member having a flow pathway, at least one inflow area communicating with the flow pathway, and an outflow area communicating with the flow pathway. The elongate member includes at least one porous structure and is configured to transition between a compressed shape when compressed and an expanded shape when uncompressed. The elongate member is also configured to be positioned in an eye such that the inflow area communicates with an anterior chamber of the eye and the outflow area communicates with a region of the eye that will increase aqueous outflow to help maintain a proper pressure of the eye.

### BRIEF DESCRIPTION OF THE DRAWINGS

FIG. 1 is a cross-sectional, perspective view of a portion of the eye showing the anterior and posterior chambers of the eye.

FIG. 2 is a cross-sectional view of a human eye.

FIG. 3A shows a first embodiment of an implant 100 in an expanded state.

FIG. 3B shows a second embodiment of an implant 200 with a lumen 214 in an expanded state.

FIG. 3C shows a third embodiment of an implant 300 with a lumen 314 in an expanded state.

FIG. 3D shows a fourth embodiment of an implant 400 with a single component porous body 420 in an expanded state.

FIG. 3E shows a fifth embodiment of an implant 500 with an internal solid member 510 and an open-celled porous layer 520 in an expanded state.

FIG. 3F shows a sixth embodiment of an implant 600 without a lumen in an expanded state.

FIGS. 4A-4C show one embodiment of a method for delivering an implant.

FIGS. 5A-5D show different implant designs 1100, 1200, 1300, and 1400 in their compressed and expanded states.

FIG. 6 shows a seventh embodiment of an implant 700.

FIGS. 7A-7D show another embodiment of an implant delivery method.

FIG. 8 shows a cross-sectional view of the eye.

FIG. 9 shows at least one implant mounted into a delivery system 800 for delivery into the eye.

FIGS. 10A-10B show enlarged views of the anterior region of the eye.

FIG. 11 shows the implant that has been placed into the eye.

FIGS. 12A-12B shows a delivery instrument 800.

FIGS. 13A-13B show another embodiment of delivery instrument 800.

### DESCRIPTION

Disclosed are devices and methods for treatment of eye disease such as glaucoma. An implant is placed in the eye wherein the implant provides a fluid pathway for the flow or drainage of aqueous humor from the anterior chamber to the supraciliary or the suprachoroidal space, or to any space in the eye where drainage to that location will lower the intraocular pressure. The implant may include an elongate compressible structure and may be implanted in the eye using a delivery system that folds and or compresses the implant to provide a smaller cross-sectional area to allow a more minimally-invasive procedure. The compressibility of the implant is provided by a porous structure that may be collapsed by compression and delivered through a tube-shaped introducer. The delivery tube need not have a round cross section, and almost any cross-sectional shape for the delivery tube may be used, i.e. oval or square cross section shapes are possible. The collapsed implant structure recovers and expands to a larger cross-sectional area after it is introduced into the eye and the compression is removed. This may be achieved by placing the compressed implant inside a delivery tube to surgically deliver it to a desired location in the eye, and then retracting the delivery tube against a stop that prevents the retraction of the implant along with the retraction of the delivery tube, thereby causing it to be released from the delivery tube end and to expand in a precise surgical location inside the eye. Other methods of implanting and expanding the implant are possible, and this example is not meant to exclude these other possible delivery methods in any way.

The porous structure of the implant may be either an open-cell or a closed-cell type of structure, or a mixture of these types, to direct the draining aqueous humor either distally or laterally, or both, through the elongate implant. Open-cell structures may include structures where each pore allows the flow of liquid into almost every adjacent pore. Closed-cell structures may include structures where each pore does not allow the flow of liquid into almost every adjacent pore. Thus, in open-cell structures there is a communication between the cells or pores that generally results from the pores being interconnected by some area of openness between adjacent cells or adjacent pores.

Thus, closed-cell structures may not be permeable to fluids passing through them and open-cell structures may be permeable to fluids passing through them. The porous structure of the implant may also be used to provide a scaffolding for tissue ingrowth, prevent implant migration, carry lubricants, or carry and release drugs into the implantation site. The pore size distribution of the device may be tuned and selected to create the proper environment for tissue ingrowth, for avoidance of excessive fibrous scarring, and for the prevention of migration after surgical placement in tissue. Layered or otherwise heterogeneous structures may be used in the implant, either made from materials with different types (open-cell vs. closed-cell vs. non-porous solid) or from different degrees of porosity (material mixed that have different pores sizes in them). Both homogenous and heterogeneous design of the structures may be used to tune the mechanical properties of the implant, to allow it to be compressed during implantation, to expand or change shape when the compression is removed, and to resist any collapse or pinching of the elongate structure that would prevent its ability to transfer fluid from one location in the eye to another.

Typically, for optimum tissue ingrowth and avoidance of excessive tissue scarring, an open-cell porous structure with a narrow distribution of pore sizes between 30 and 40 microns may be used, at least in the outer, external surface areas of the implant that will directly contact tissue after the implant is placed into tissue. This pore size distribution allows tissue to grow into the outer pores without excess fibrosis and scarring, and allows the inner volume of open-cell pore structures to conduct fluid flow through the device without interference from fibrosis and scarring.

The elongate implant may shunt fluid from one location to another location in the eye, may be compressed to be surgically implanted at a smaller cross-sectional dimension, and may have a surface porosity configured to prevent excess fibrosis and scarring. The elongate implant may include an outer layer of surface porosity, to provide for the ingrowth of tissue into the outer surfaces of the implant after the implant is released and expanded inside the eye, to help prevent the implant from migrating in tissue after it is surgically placed. the elongate implant may include an outer layer of surface porosity, to provide an improved holding power in the tissue after the implant is released and expanded inside the eye, to help prevent the implant from migrating in tissue after it is surgically placed.

Depending on the areas of the implant where permeability is desired, a layer of closed-cell foam underneath or above the open-cell foam structure may be used to provide a collapsible structure that is non-permeable in specific regions. Alternatively, a thin, non-permeable wall structure under an open-cell foam structure may be used to prevent permeable outflow from the implant in selected locations. In such an embodiment, the wall thickness of the solid material may be small to allow this part of the implant structure to fold and collapse under compression to a smaller cross-sectional area. Still alternatively, wall structures that contain layers of open cell foam over layers of non-permeable, closed-cell foam, over layers of non-permeable solid walls are possible to tune the permeability, the shape change response to compression, and the device response to bending and kinking inside the eye. Many layer combinations of the solid wall or bar, open- and closed-cell foam layer structures may be used to tune the mechanical behavior of the implant, such as foam sandwich constructions which are stiff yet lightweight.

Any combination of permeable and non-permeable foam and solid materials may be used to make the implant as a heterogeneous, composite structure and the implant is not limited to using any specific combination except to provide a foldable or collapsible structure that may be introduced into the eye at a smaller cross section area than it will have when it changes shape to a larger cross section after it is released from the surgical introducing instrument. Thus, an implant made from a single type of foam, such as an elongate closed-cell foam tube with an internal lumen, may be used. In a different embodiment, an open-celled, foam elongate cylinder without an internal lumen or an elongate non-cylindrical bar without an internal lumen may be used. Both of these implant designs may be compressible to a smaller diameter or cross-sectional area, and both may allow a flow of fluid to occur through the elongate length of the implant to drain excess aqueous humor.

The implant described herein is designed to enhance aqueous flow through the normal outflow system of the eye while reducing complications. The structure may be inserted in a constrained configuration that reduces the cross sectional area of the implant and may recover to an expanded cross-sectional shape after implantation in the eye to enhance retention of the device in the eye as well as improve fluid flow. Any of the procedures and devices described herein may be performed in conjunction with other therapeutic procedures, such as laser iridotomy, laser iridoplasty, and goniosynechialysis (a cyclodialysis procedure).

In one embodiment, disclosed is an ocular implant including an elongate member having at least one flow pathway, at least one inflow area communicating with the flow pathway and at least one outflow area communicating with the flow pathway. The elongate member may include a portion formed from an open-cell porous cylinder or open-cell porous non-cylindrical bar without an internal lumen and is adapted to transition between a first shape when in compression and a second shape upon release of compression. The elongate member is adapted to be positioned in the eye such that the inflow area communicates with the anterior chamber and the outflow area communicates with any area of the eye where drainage through the device may lower intraocular pressure in the eye, such as the supraciliary or suprachoroidal spaces, Schlemm's canal, intrascleral pockets or spaces, scleral veins, or the subconjuctiva.

In one embodiment, disclosed is an ocular implant including an elongate member having at least one flow pathway, at least one inflow area communicating with the flow pathway and at least one outflow area communicating with the flow pathway. The elongate member includes a first portion formed from an open-cell porous cylinder or open-cell porous non-cylindrical bar and a second portion formed from a non-porous internal solid bar and is adapted to transition between a first shape when in compression and a second shape upon release of compression. The elongate member is adapted to be positioned in the eye such that the inflow area communicates with the anterior chamber and the outflow area communicates with any area of the eye where drainage through the device may lower intraocular pressure in the eye, such as the supraciliary or suprachoroidal spaces, Schlemm's canal, intra scleral pockets or spaces, scleral veins, or the subconjuctiva.

In one embodiment, disclosed is an ocular implant including an elongate member having at least one flow pathway, at least one inflow port communicating with the flow pathway and at least one outflow port communicating with the flow pathway. The inflow and outflow ports may or may not be a completely open area, alternatively they may be covered with an open cell foam that allows fluid to pass through the foam in these ports. The elongate member includes a first portion formed from at least a partially porous wall or foam structure and adapted to transition between a first shape when in compression and a second shape upon release of compression and a second portion formed at least partially of a non-fluid permeable structure, either a non-porous, bulk (or solid) material or non-permeable, close cell foam, or both. The elongate member is adapted to be positioned in the eye such that the inflow port communicates with the anterior chamber and the outflow port communicates with any area of the eye where drainage through the device may lower intraocular pressure in the eye, such as the supraciliary or suprachoroidal spaces, Schlemm's canal, intrascleral pockets or spaces, scleral veins, or the subconjuctiva.

In another embodiment, disclosed is an ocular implant including an elongate member having a flow pathway, at least one inflow port communicating with the flow pathway, and at least one outflow port communicating with the flow pathway. At least a portion of the elongate member is adapted to reversibly deform between a first shape and a second shape upon release of compression. The elongate member is adapted to be positioned in the eye such that the inflow port communicates with the anterior chamber and the outflow port communicates with any outflow drainage spaces mentioned above.

In an embodiment, disclosed is a method of implanting an ocular device into the eye. The method includes forming an incision in the cornea of the eye; inserting an implant having a fluid passageway through the incision into the anterior chamber of the eye while the implant is under compression. The compression maintains the implant in a first shape. The method also includes the steps of passing the implant along a pathway from the anterior chamber into the supraciliary and or suprachoroidal space; positioning the implant in a first position such that a first portion of the fluid passageway communicates with the anterior chamber and a second portion of the fluid passageway communicates with the supraciliary and or suprachoroidal space to provide a fluid passageway between the supraciliary and or suprachoroidal spaces and the anterior chamber; and releasing the implant from compression wherein the release of compression permits the implant to transition to a second shape. Generally, it is desirable that the first shape held under compression has a smaller cross sectional area then the cross sectional area of second shape when compression is removed. This smaller cross-sectional shape allows the device to be implanted through a smaller incision with less likelihood of rubbing sensitive eye tissues during implantation.

In another embodiment, disclosed is a method of implanting an ocular device into the eye that includes the steps of forming an incision in the cornea of the eye; loading into the compression tube or cannula of a delivery device a compressed, reduced cross sectional area implant. The delivery tube is adapted to impose compression to deform at least a portion of the implant into a first, smaller cross section shape to allow a less invasive implantation through the anterior chamber and into the supraciliary and or suprachoroidal space. Once the implant is in the desired location, it is released from the compression tube to change into a second shape conducive to implant retention and flow function. The method also includes the steps of inserting the implant loaded in the compression tube through the incision into the anterior chamber of the eye; passing the implant along a pathway from the anterior chamber into the supraciliary and or suprachoroidal space; positioning the implant in a first position such that a first portion of the fluid passageway communicates with the anterior chamber and a second portion of the fluid passageway communicates with the supraciliary and or suprachoroidal spaces to provide a fluid passageway between the anterior chamber and one or more of these spaces; and releasing the implant from the delivery device wherein the release removes the compression and permits at least a portion of the implant to change to the second shape conducive to retention of the device in tissue and flow through the device to the supraciliary and or suprachoroidal spaces.

In one embodiment, disclosed is a system for treating an ocular disorder in a patient. The system includes an elongate member having a flow pathway, at least one inflow port communicating with the flow pathway, and at least one outflow port communicating with the flow pathway. The elongate member includes a first portion formed of a porous foam structure and adapted to transition between a first shape when in compression and a second shape upon release of compression, and a second portion formed at least partially of a non-porous structure. The elongate member is adapted to be positioned in the eye such that the inflow port communicates with the anterior chamber and the outflow port communicates with the supraciliary and or suprachoroidal spaces; and a delivery device having a delivery component that contains the compressed elongate member and can release the elongate member to a location inside the eye. The delivery component is adapted to maintain the elongate member in compression until it is released to a location in the eye, whereupon it expands to changes shape.

In another embodiment, disclosed is a system for treating an ocular disorder in a patient. The system includes an elongate member having a flow pathway, at least one inflow port communicating with the flow pathway, and an outflow port communicating with the flow pathway. At least a portion of the elongate member is adapted to reversibly deform between a first shape and a second shape. The elongate member is adapted to be positioned in the eye such that the inflow port communicates with the anterior chamber and the outflow port communicates with the supraciliary and or suprachoroidal spaces. The system also includes a delivery device having a delivery component that couples to the elongate member. The delivery component is adapted to deform at least a portion of the elongate member into the first shape by imposing compression.

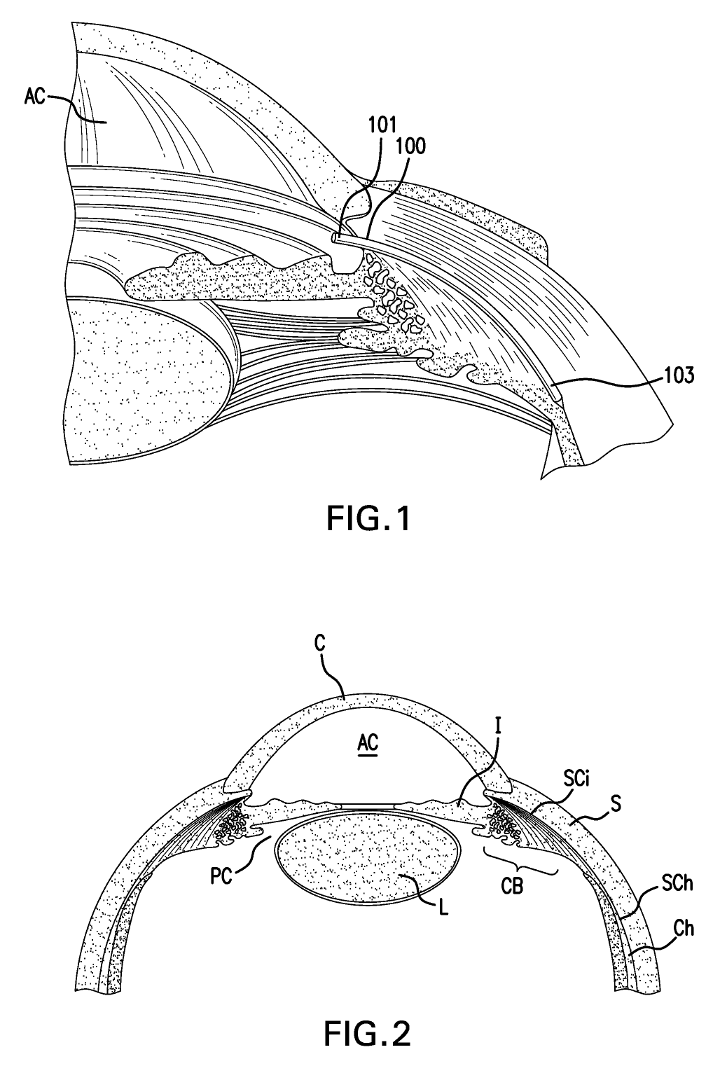

FIG. 1 is a cross-sectional, perspective view of a portion of the eye showing the anterior and posterior chambers of the eye. A schematic representation of an implant 100 is positioned inside the eye such that a proximal end 101 is located in the anterior chamber AC and a distal end 103 is located in or near the supraciliary space SCi and/or the suprachoroidal space SCh (sometimes referred to as the perichoroidal space). The suprachoroidal space SCh may include the region between the sclera and the choroid. The supraciliary space SCi may also include the region between the sclera and the ciliary body. Implant 100 may be positioned at least partially between the ciliary body and the sclera or it may be at least partially positioned between the sclera and the choroid. Implant 100 may also be at least partially positioned in the suprachoroidal space SCh. In any event, implant 100 provides a fluid pathway between the anterior chamber AC and the supraciliary space SCi and/or the suprachoroidal space SCh, depending on the length and regions of permeability of implant 100 along its length.

In one embodiment, at least a portion of implant 100 may be an elongate open cell porous element without an internal lumen, through which aqueous humor may flow from the anterior chamber AC into the supraciliary space SCi and/or the suprachoroidal space SCh, such as in the region between the sclera and the choroid. At least a portion of implant 100 may be formed of a porous structure that is adapted to change from a first shape to a second shape. The change in shape may occur prior to, during, or after the implant is implanted in the eye. Implant 100 may have a substantially uniform diameter along its entire length, although the shape of implant 100 may also vary along its length (either before or after insertion of implant 100). Moreover, implant 100 may have various cross-sectional shapes (such as circular, oval, or rectangular) and may vary in cross-sectional shape moving along its length. The cross-sectional shape may be selected to facilitate easy insertion into the eye. In one embodiment, implant 100 may be manufactured at least partially of a shape-changing material.

It should be appreciated the several shape change configurations are considered herein. It should also be appreciated that features described with respect to one embodiment may be used with other embodiments described herein.

Exemplary Eye Anatomy

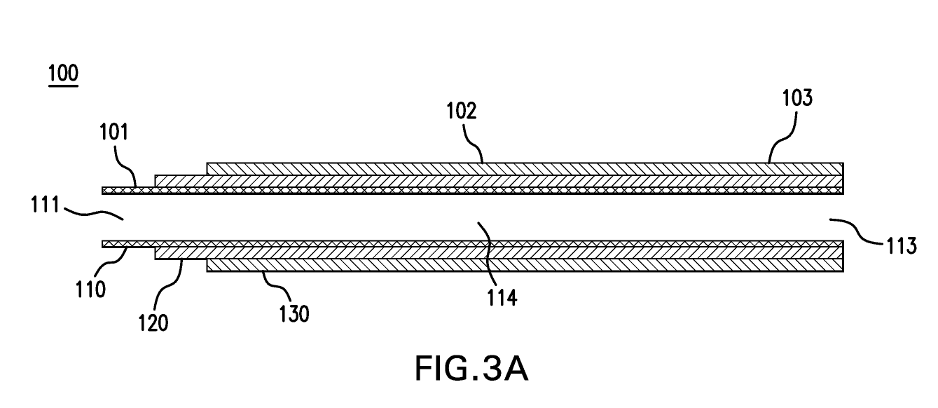

FIG. 2 is a cross-sectional view of a human eye. The eye is generally spherical and is covered on the outside by the sclera S and the cornea C. The retina lines the inside posterior half of the eye. The retina registers the light and sends signals to the brain via the optic nerve. The posterior section of the eye is filled and supported by the vitreous body, a clear, jelly-like substance. The supraciliary space SCi is the region between the ciliary body CB and the sclera S, and the suprachoroidal space SCh is the region between the sclera S and the choroid Ch.

The elastic lens L is located near the front of the eye. The lens L provides adjustment of focus and is suspended within a capsular bag from the ciliary body CB, which contains the muscles that change the focal length of the lens. A volume in front of the lens L is divided into two by the iris I, which controls the aperture of the lens and the amount of light striking the retina. The pupil is a hole in the center of the iris I through which light passes. The volume between the iris I and the lens L is the posterior chamber PC. The volume between the iris I and the cornea C is the anterior chamber AC. Both chambers are filled with a clear liquid known as aqueous humor.

The ciliary body CB continuously forms aqueous humor in the posterior chamber PC by secretion from the blood vessels. The aqueous humor flows around the lens L and iris I into the anterior chamber AC and exits the eye through the trabecular meshwork, a sieve-like structure situated at the corner of the iris I and the wall of the eye (the corner is known as the iridocorneal angle). Some of the aqueous humor filters through the trabecular meshwork into Schlemm's canal, a small channel that drains into the ocular veins. A smaller portion rejoins the venous circulation after passing through the ciliary body CB and eventually through the sclera S. This outflow path is known as the uveoscleral outflow path.

Glaucoma is a disease wherein the inflow and outflow of aqueous humor is not properly balanced and pressure builds up within the eye. In a healthy eye, the ciliary processes secrete aqueous humor, which then passes through the angle between the cornea C and the iris I. Glaucoma appears to be the result of clogging in the trabecular meshwork. The clogging can be caused by the exfoliation of cells or other debris. When the aqueous humor does not drain properly from the clogged meshwork, it builds up and causes increased pressure in the eye, particularly on the blood vessels that lead to the optic nerve, which can result in death of retinal ganglion cells and eventual blindness.

Closed angle glaucoma can occur in people who were born with a narrow angle between the iris and the cornea (the anterior chamber angle). This is more common in people who are farsighted (they see objects in the distance better than those which are close up). The iris can vault forward and close off or restrict the exit of aqueous humor, and an increase in pressure within the eye occurs.

Open angle glaucoma is by far the most common type of glaucoma. In open angle glaucoma, the iris does not block the drainage angle as it does in closed angle glaucoma. Instead, the fluid outlet channels of the eye gradually narrow with time and lose their capacity to drain enough of the aqueous humor. The disease usually affects both eyes, and over a period of years the consistently elevated pressure slowly damages the optic nerve.

Embodiments of Shape-Change Retention Implants

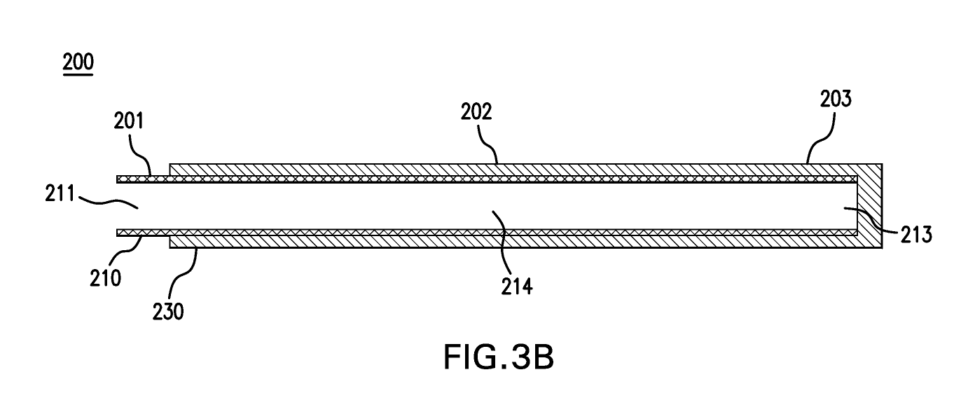

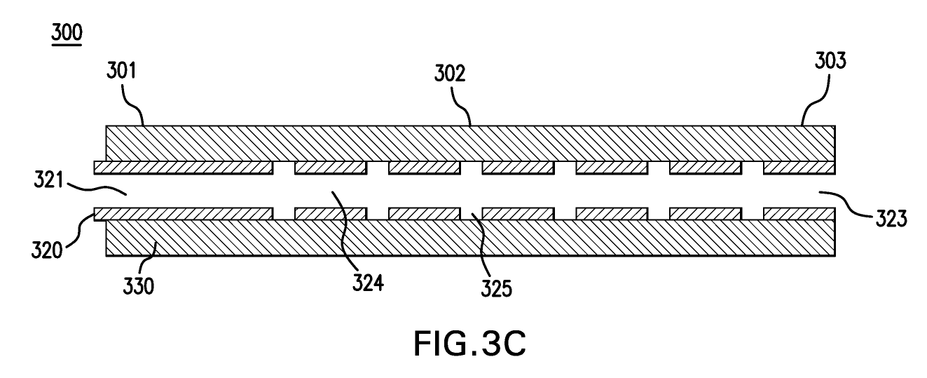

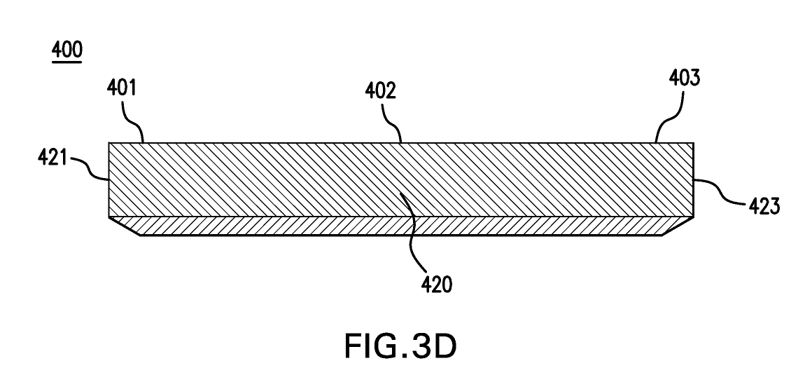

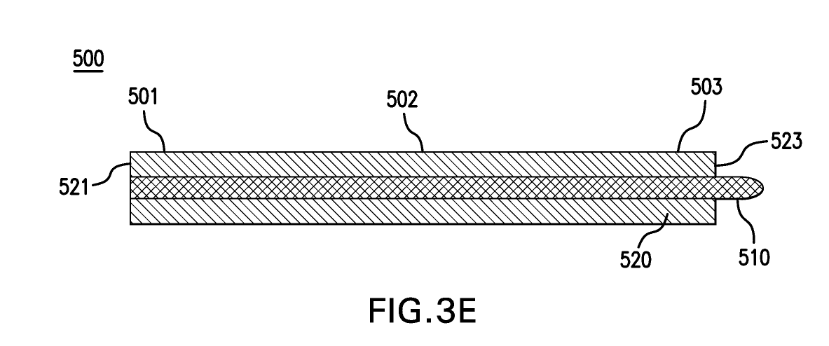

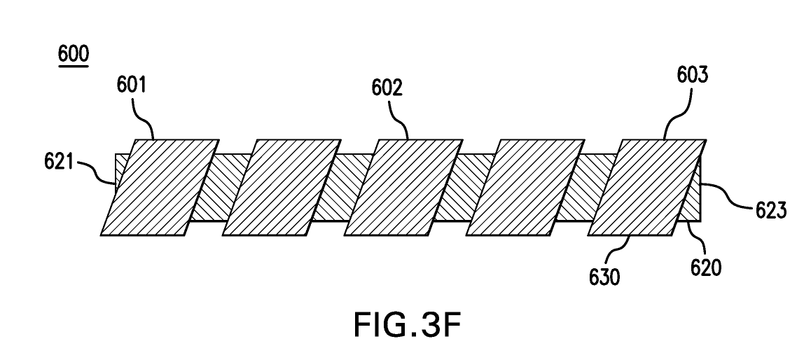

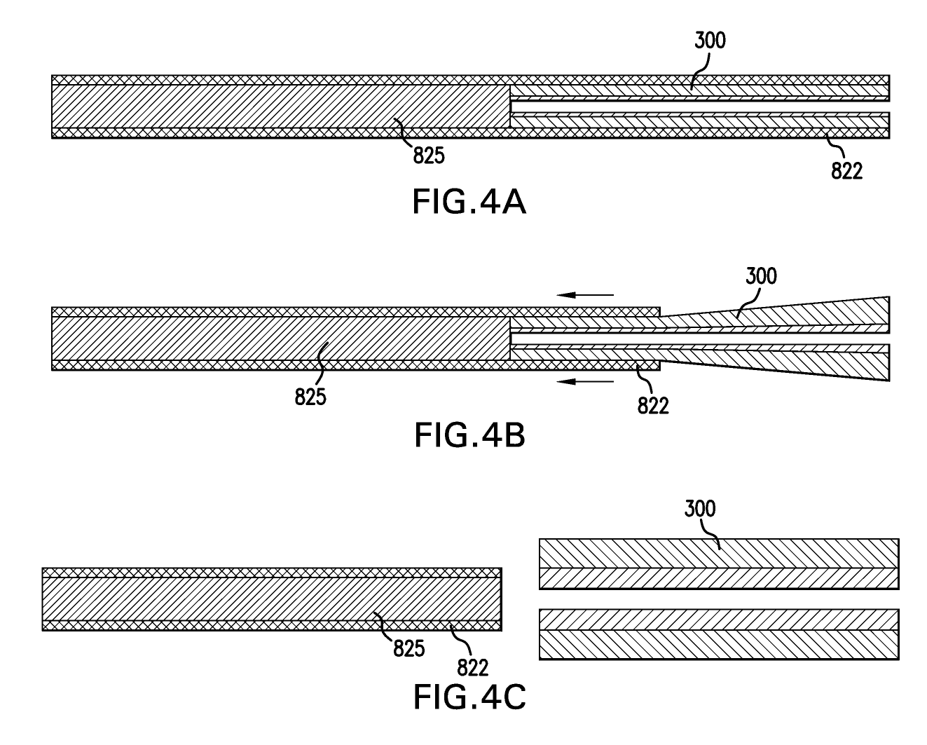

FIG. 3A shows a first embodiment of an implant 100 in an expanded state. Implant 100 may be an elongate member having a proximal portion 101, a distal portion 103, and a structure that permits fluid (such as aqueous humor) to flow along the length of implant 100, such as through implant 100. Implant 100 may include at least one lumen 114 having at least one proximal opening 111 for ingress of fluid (such as aqueous humor from the anterior chamber) and at least one distal opening 113 for egress of fluid. Implant 100 may include various arrangements of openings that communicate with lumen 114.

Lumen 114 serves as a passageway for the flow of aqueous humor through implant 100 directly from the anterior chamber AC to the supraciliary space SCi and/or the suprachoroidal space SCh. In addition, lumen 114 may be used to mount implant 100 onto a delivery system. Implant 100 may have a substantially uniform diameter along its entire length, although the diameter of implant 100 may vary along its length (either before or after expansion of the implant). Moreover, implant 100 may have various cross-sectional shapes (such as circular, oval, or rectangular) and may vary in cross-sectional shape moving along its length. The cross-sectional shape, the wall morphology type (open-cell material vs. closed-cell material vs. solid material), and geometric shape and pattern of the layers may be selected to facilitate insertion into the eye and to keep lumen 114 from kinking when it is bent inside of the eye.

Implant 100 may include a multi-layered tubular or partially tubular structure. Implant 100 may be at least partially manufactured of a porous structure formed of polymeric or metal materials that are biocompatible. The porous structures may be arranged in layers and each layer may be a patterned to have holes or openings in that layer of various shapes. One or more porous structured layers may be positioned over or otherwise combined with a solid tube or with a closed cell foam tube wherein the solid tube or the closed cell foam tube has an internal lumen through which fluid may travel. Thus, the porous structure(s) and the solid tube collectively may form a layered structure and, depending on the patterning of openings between the layers, the control of longitudinal vs. lateral flow of aqueous humor through implant 100 may be fine tuned.

Implant 100 may include a proximal portion 101, a central portion 102, and a distal portion 103. Proximal portion 101 may include a solid tube 110 alone (with lumen 114), or a solid tube 110 that is over layered with a porous layer 130 such that the proximal portion 101 is either a single- or a multi-layered portion. Central portion 102 may include three layers: a solid inner tube 110, a middle non-permeable closed-cell porous layer 120, and an outer permeable porous layer 130. Alternatively, central portion 102 may include a solid inner tube 110 over layered with a permeable porous layer 130. Distal portion 103 may include both closed-cell porous layer 120 and open-cell porous layer 130.

Central portion 102 may also allow fluid flow if holes are created in the layers that are impermeable to fluid flow, that is if holes are created in inner solid wall 110 and middle closed pore wall 120 to allow fluid to escape through these holes. Thus, fluid may flow through the openings in an unimpeded manner. The openings in the non-permeable walls may be over layered with a permeable porous material whose pore size distribution and pore dimensions are tuned to allow tissue in growth into the outer layer with out excessive scarring when the device is implanted in the eye. The porous material of implant 100 may also be filled with a drug or treated with a biocompatible lubricant to allow it to be expanded from the delivery device with out excessive friction between the delivery tube and the compressed implant 100.

FIG. 3B shows a second embodiment of an implant 200 with a lumen 214 in an expanded state. An inner layer 210 may be made from a thin solid material and an outer layer 230 may be made from an open cell porous layer that may be compressed to a smaller cross-sectional area dimension. Implant 200 may be an elongate member having a proximal portion 201, a central portion 202, and a distal portion 203, and a structure that permits fluid (such as aqueous humor) to flow along the length of implant 200 such as through lumen 214 of implant 200. Implant 200 may include at least one lumen 214 having at least one proximal opening 211 for ingress of fluid (such as aqueous humor from the anterior chamber) and at least one distal opening 213 for egress of fluid. Implant 200 may include a lumen 214 whose distal opening 213 is entirely covered with permeable porous layer 230. The open-cell porous material covering distal opening 213 allows fluid to escape through distal opening 213 since this covering is permeable.

FIG. 3C shows a third embodiment of an implant 300 with a lumen 324 in an expanded state. An inner layer 320 may be made from a closed-cell material and an outer layer 330 is made from an open-cell porous layer that may be compressed. Implant 300 may be an elongate member having a proximal portion 301, a central portion 302, and a distal portion 303, and a structure that permits fluid (such as aqueous humor) to flow along the length of implant 300 such as through lumen 324 of implant 300. Implant 300 may include at least one lumen 324 having at least one proximal opening 321 for ingress of fluid (such as aqueous humor from the anterior chamber) and at least one distal opening 323 for egress of fluid. Inner layer 320 may include holes 325 that allow fluid to escape from lumen 324 into outer layer 330, which is permeable since it is made from an open-celled porous material. Since inner and outer layers 320 and 330 may be made from porous material, implant 300 may be compressed to a smaller cross-sectional area dimension.

FIG. 3D shows a fourth embodiment of an implant 400 with a single component porous body 420 in an expanded state. Main body 420 may be made from an open-cell porous material that may be compressed to a smaller cross-sectional area dimension. Implant 400 may be an elongate member having a proximal portion 401, a central portion 402, and a distal portion 403, and a structure that permits fluid (such as aqueous humor) to flow along the length of implant 400 such as through the open-celled porous body 420 of implant 400. Implant 400 may include at least one inflow area 421 for ingress of fluid (such as aqueous humor from the anterior chamber) and at least one outflow area 423 for egress of fluid.

FIG. 3E shows a fifth embodiment of an implant 500 with an internal solid member 510 and an open-celled porous layer 520 in an expanded state. Internal solid member 510 may be made from a relatively stiffer solid or stiffer porous material and outer body 520 may be made from an open-cell porous layer that may be compressed. Implant 500 may be an elongate member having a proximal portion 501, a central portion 502, and a distal portion 503, and a structure that permits fluid (such as aqueous humor) to flow along the length of implant 500 such as through the open-celled pores of body 520 of implant 500. Implant 500 may include at least one inflow area 521 for ingress of fluid (such as aqueous humor from the anterior chamber) and at least one outflow area 523 for egress of fluid. Since outer body 520 may be made from porous material, implant 500 may be compressed to a smaller cross-sectional area dimension. Inner member 510 acts as a “spine” for implant 500 to add to its stiffness and to act as a memory shape, such as a pre-set curvature along the length of implant 500. Inner member 510 may be made from a solid material or from a stiffer porous material, such as a porous ceramic or porous metal, but it need not be compressible, since it may be of a small cross-sectional area. Inner member 510 may also extend beyond outflow area 523 of porous body 520 of implant 500 and be used to help separate or dissect tissue when implant 500 is being surgically implanted into the eye. Inner member 510 may alternatively be a smaller diameter, stiff polymeric or metal tube. The cross-sectional area of member 510 may be varied, such as with ridges or flanges, as long as the largest cross-sectional area of member 510 does not prevent outer body 520 of implant 500 from being at least partially compressed to a smaller cross-sectional area.

FIG. 3F shows a sixth embodiment of an implant 600 without a lumen in an expanded state. An inner porous body 620 may be made from an open-cell material and an outer spiral winding 630 may be made from a closed-cell porous layer that wraps around inner body 620. Implant 600 may be an elongate member having a proximal portion 601, a central portion 602, and a distal portion 603, and a structure that permits fluid (such as aqueous humor) to flow along the length of implant 600 such as through the open cell porous structure of inner body 620. Implant 600 may include at least one inflow area 621 for ingress of fluid (such as aqueous humor from the anterior chamber) and at least one outflow area 623 for egress of fluid. Outer layer 630 may be a spiral that is not permeable, but outer layer 630 may include gaps or spaces between the spirals that also allow fluid to escape laterally from internal body 620 in the proximal, middle, and distal portions 601, 602, and 603 where the gaps are present. Since the inner body 620 and outer spiral layer 630 may be made from porous materials, implant 600 may be compressed to a smaller cross-sectional area dimension. Implant 600 may have a variable cross-sectional thickness in the expanded state due to spiral layer 630 extending beyond the cross-sectional thickness of inner body 620. By varying the geometry of inner body 620 and outer layer 630, the mechanical properties of implant 600 may be tuned to adjust both its bending behavior to resist kinking or collapsing and fluid flow characteristics (longitudinal flow volume vs. lateral flow volume).

Shape Changing by Compression and Expansion

The layered porous wall structure of the implant is configured to change shape, such as to expand the cross-sectional area of the implant, during or after implantation in the eye. Importantly, since the device is compressed radially, without stretching it longitudinally, the expansion of the device cross section occurs largely or entirely without a change in the length of the implant, a considerable advantage over other shape changing structures, such as braided structures, when locating the device in the eye surgically. The shape change may facilitate anchoring in the eye and prevent migration of the implant once it is positioned in the eye. The areas of the implant that are permeable and allow outflow from selected areas of the implant may be positioned so as to align with predetermined anatomical structures of the eye. For example, one or more fully permeable openings may align with the suprachoroidal space to permit the flow of aqueous humor into the suprachoroidal space, while another set of fully permeable openings may align with structures proximal to the supraciliary space, such as structures in the ciliary body, and finally a set of fully permeable openings may align with the location of the proximal portion placed in the anterior chamber to allow aqueous humor to flow from the anterior chamber of the eye. The term “fully permeable openings” may describe areas or volume sections of the implant where there are no non-permeable barriers for flow through the walls or through these volume sections of the device that would completely prevent fluid flow in these areas. These areas may or may not be covered with an additional permeable porous layer.

The shape change may occur in a variety of manners, but it is primarily by the compression of a porous structure that is compressed to collapse the pores in the volume of the structure (and increase the bulk material density) and then releasing the compression to allow the collapsed structure to spring open and re expand the pores (and re establish a lower bulk material density). The change in density that occurs during the shape change allows the implant to be compressed with little or no change in length that would normally result from the Poisson effect from an incompressible or nearly incompressible solid. The increase in density of the collapsed implant that results from the collapse of the pores in the structure may also be beneficial to help the implant penetrate the tissue during surgical implantation while the implant is compressed, since the increased implant density from compression will support greater longitudinal stress compared to when the implant is expanded and at a lower bulk density. Thus, the increase in density may allow the compressed implant to be advanced more easily through tissue or even make it stiff enough to dissect or separate tissue when it is pushed into tissue. Implants that shape change with significant changes in length, such as braided structures, may be difficult to locate accurately in tissue when they are surgically delivered and as they are released and expanded.

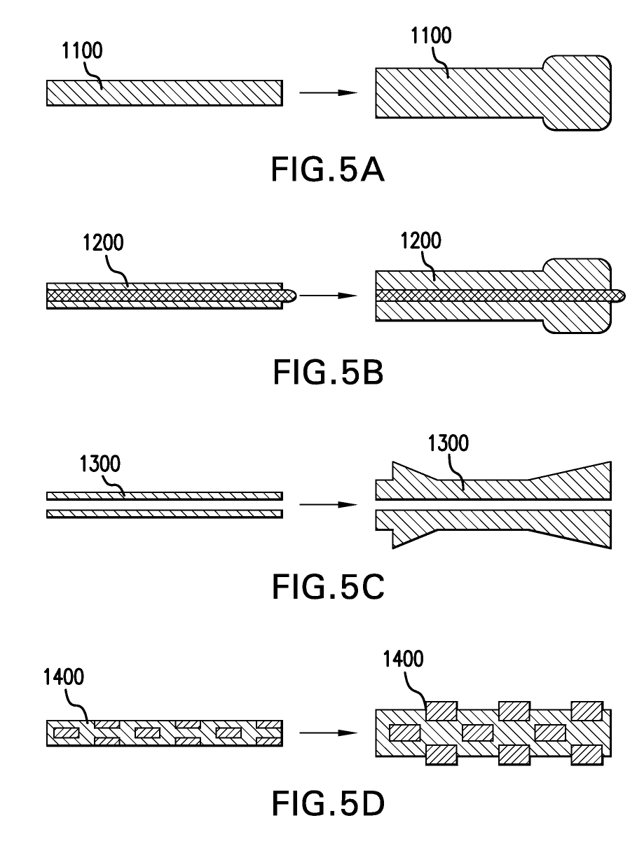

Referring to FIG. 4A, implant 300 is compressed inside a delivery tube 822 and rests against a stop 825 at proximal end 301 of implant 300. As shown in FIG. 4B, as delivery tube 822 is retracted the compressive forces on implant 300 are removed and implant 300 expands to take an expanded shape. As shown in FIG. 4C, the compressed implant 300 is ejected from delivery tube 822 by retracting tube 822 over stop 825, which prevents proximal end 301 of implant 300 from moving with the retracted tube 822. The expanded shape may not significantly alter the length of implant 300 after it is expanded so that the length of implant 300 remains nearly or completely identical. Delivery tube 822 may be flexible and compliant enough to allow for blunt dissection such as between the tissue layers of the sclera and ciliary body and able to follow the natural curve of the inner scleral wall. Delivery tube 822 may also be used with any other suitable implant.

Additional Implant Features

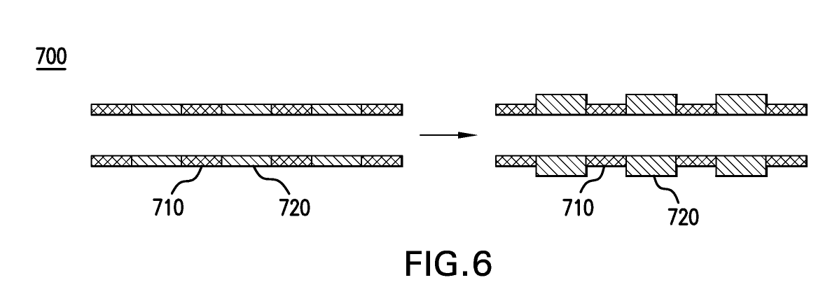

The implants described herein may include additional features to improve their effectiveness in draining fluid from the anterior chamber to the supra ciliary or suprachoroidal spaces. The implants described herein may also include additional structural features in addition to the shape change region that assist in anchoring or retaining the implant in the eye. For example, the implants described herein may be equipped with non-uniform expanded cross-sectional areas at the proximal or distal end or anywhere along the length of the implant to help further the retention strength in the tissue, or to help open areas of tissue surrounding the implant. Several designs used to illustrate this idea are shown in FIGS. 5A-5D. In FIGS. 5A-5D, four different implant designs 1100, 1200, 1300, and 1400 are shown in their compressed and expanded states. Implant 1100 is a single-element open-celled porous implant. Implant 1200 is a single-element open-celled porous implant with an internal solid member. Implant 1300 is a single-element open-celled porous implant with an internal lumen. Implant 1400 is a dual-element porous implant with closed-cell porous nubs. In the compressed state the implants are all compressed to fit inside delivery tube 822. Once the implants are released from delivery tube 822 they expand. The expanded implants may have variable cross-sectional areas along their elongate dimension that may be used to help retain the implants in place (prevent migration in the tissue) and to open up additional tissue areas surrounding the implant in selected tissue locations.

The implant may include one or more retaining or retention structures, such as flanges, protrusions, wings, tines, or nubs, that extend into the surrounding eye anatomy to retain the implant in place and prevent the implant from moving. The retention features may also provide regions for fibrous attachment between the implant and the surrounding eye anatomy. This is particularly true for open-cell porous structure of the proper pore size and pore size distribution. Many possible combinations of porous structural protrusions are possible to create the variable cross section features when the implants are delivered and expanded in the tissue and these examples are not meant to be limiting in any way.

The change in shape may be an outward, radial expansion, or it may be combined with other changes in shape, such as a change from a straightened to a non-straightened (e.g., curved or wavy) elongate shape, but the shape change may avoid any significant change in the length or arc length of the implant, particularly in any sections of the implant that are designed to expand and increase in cross-sectional area. The implants described herein may also include designs where only some sections of the implant may undergo shape change, while other sections may not undergo shape change. The shape changes that occur to increase the cross sectional area of the implant in certain regions along the length of the implant may not result in a significant change of length of the implant in the shape-changing regions when those regions expand.

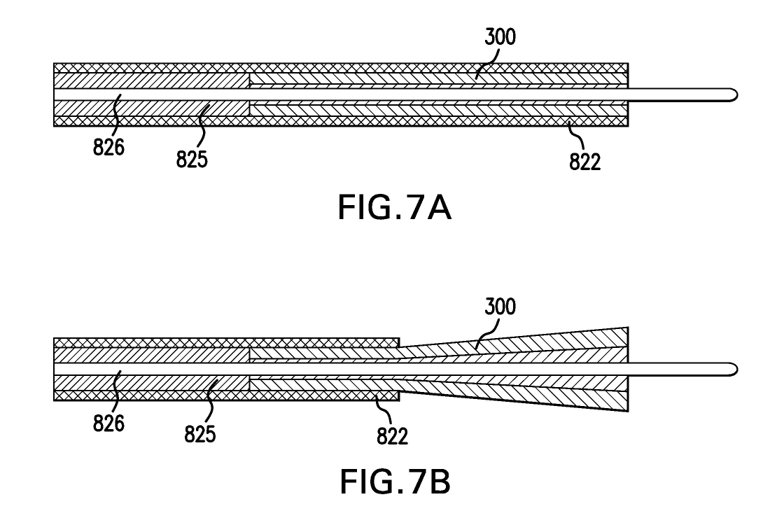

Referring to FIG. 6, a seventh embodiment of an implant 700 is shown. Implant 700 includes regions 720 that shape change to expand their cross sectional area when compression is removed, and regions 710 that do not undergo any shape change when compression is removed. Many different combinations of shape-changing regions 720 and non-shape-changing regions 710 are possible, and the example in FIGS. 6A-6B is not meant to be limiting to other embodiments of an elongate implant that may consist of both expandable regions 720 and non-expandable regions 710.

The shape change of the porous implants of this application may not require bodily fluids or water to expand the material, and any water that is adsorbed into the open celled porous structure may be rapidly squeezed out, unlike structures that expand primarily by absorbing water into the free space of the bulk of the polymer, such as with solid hydrogel polymers or gels. Although water may be removed from solid hydrogel polymers, they are not very compressible and once hydrated, they are typically dehydrated by methods other than compression to reduce their volume without damaging the polymer. One embodiment of an implant may include porous hydrogel structures that could be mechanically collapsed and expanded by virtue of there being open- or closed-cell pores within the hydrogel structure. Since hydrogels are very mechanically weak, the use of hydrogels that contain higher strength interpenetrating network (IPN) of polymers to reinforce the hydrogels may be desirable, especially when making the hydrogels porous. The use of the reinforcing IPN may allow the porous hydrogel structures to have enough strength to allow the pores to spring back and recover from compression. Alternatively, a high strength open-celled porous material may be treated with a solution designed to coat some or all of the internal or external porous structures with a very thin layer of hydrogel. Such a treatment may still allow the structure to be collapsed and expanded independently of water adsorption by the hydrogel, by applying and removing the appropriate compressive forces as described in this application.

The implants described herein may have one or more features that aid in properly positioning the implant in the eye. For example, the implants may include one or more fluorescent, visible light, tomographic, radiopaque, echogenic, or infrared markers along the length to assist the user in positioning the desired portion of the implant within the anterior chamber and the desired portion within the supraciliary space during or after surgery. In using the markers to properly place the implant, the implant may be inserted in the supraciliary space, until the marker is aligned with a relevant anatomic structure, for example, visually identifying a marker on the anterior chamber portion of the implant that aligns with the proper location on the iris root, or scleral spur, such that an appropriate length of the implant remains in the anterior chamber. Under ultrasound, an echogenic marker may signal the placement of the device within the supraciliary space. Any marker may be placed anywhere on the device to provide feedback to the user on real-time placement, confirmation of placement or during patient follow up. Further, the implants and delivery system may employ alignment marks, tabs, slots or other features that allow the user to know alignment of the implant with respect to the delivery device.

Shape Change of Implant

The implants described herein may be configured to change shape, such as to bow or expand outward, during or after implantation in the eye. The material of the implants may be reversibly compressed such that it may take on a narrow profile (e.g. such as shown in the compressed implants in FIGS. 5A-5D) that is suitable for insertion through a small opening and then return to the expanded shape (e.g. such as shown in the expanded implants in FIGS. 5A-5D). The implant maintains the insertion shape when it is under compression inside a delivery tube 822 that is part of a surgical delivery instrument. When the implant is at or near the desired location in the eye inside the surgical delivery device, delivery tube 822 is retracted, but the proximal implant end may be held against a stop 825, so that the implant is ejected from tube 822 and reverts back to a expanded retention shape. Importantly, there may be little change of the length of the implant when it is ejected from delivery tube 822, allowing for accurate placement of the expanded implant in the desired tissue location.

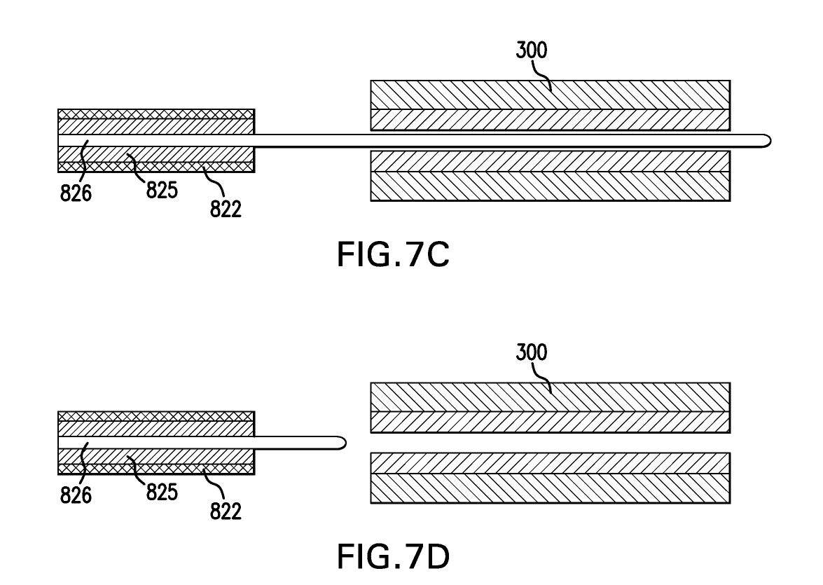

Referring to FIGS. 7A-7D, another embodiment of an implant delivery method is shown that also includes an elongate delivery wire 826 that is sized and shaped to be inserted longitudinally through the lumen of the implant. The implant delivery method may be used with implant 300, as illustrated, or any other suitable implant. In FIG. 7A, an implant 300 is located inside delivery tube 822 and rests against stop 825 at proximal end 301 of implant 300. As shown in FIG. 7B, as delivery tube 822 is retracted the compressive forces on implant 300 are removed and it expands. As shown in FIG. 7C, the compressed implant 300 is ejected from delivery tube 822 by retracting tube 822 over stop 825, which prevents proximal end 301 of implant 300 from moving with the refracted tube 822. As shown in FIG. 7D, after the retraction of delivery tube 822, guide wire 826 may be retracted, leaving expanded implant 300 in the desired location in the tissue. Alternatively, both guide wire 826 and delivery tube 822 may be retracted simultaneously to release and expand the implant 300. The expanded shape may not significantly alter the length of implant 300 after it is expanded so that the length of implant 300 remains nearly or completely identical.

Referring again to FIGS. 7A-7D, delivery wire 826 and delivery tube 822 may be more rigid than implant 300 such that they may help constrain implant 300 in the same longitudinal shape of tube 822 and wire 826. The shape of tube 822 and wire 826 do not have to necessarily be the same longitudinal shape. An example, not meant to be limiting, would be that wire 826 has a longitudinal radius of curvature that is smaller than the longitudinal radius of curvature of delivery tube 822. In this case, the radius of curvature of the implant may be made to change as delivery tube 822 is retracted, and then change again as the wire 826 is retracted. Many combinations of this two stage shape change mediated by the shapes of delivery tube 822 and guide wire 826 are possible. Both delivery tube 822 and guide wire 826 may be flexible and compliant enough to allow for blunt dissection such as between the tissue layers of the sclera and ciliary body and able to follow the natural curve of the inner scleral wall.

Exemplary Methods of Delivery and Implantation

An exemplary method of delivering and implanting the implant into the eye is now described. The method may be used with implant 300, as illustrated, or any other suitable implant. In general, the implant is implanted using a delivery system by entering the eye through a corneal or limbal incision and penetrating the iris root or a region of the ciliary body or the iris root part of the ciliary body near its tissue connection with the scleral spur to create a minimally-invasive blunt dissection in the tissue boundary between the inner scleral wall and the outer ciliary body. The implant is then positioned in the eye so that it provides fluid communication between the anterior chamber and the supraciliary and or the suprachoroidal spaces.

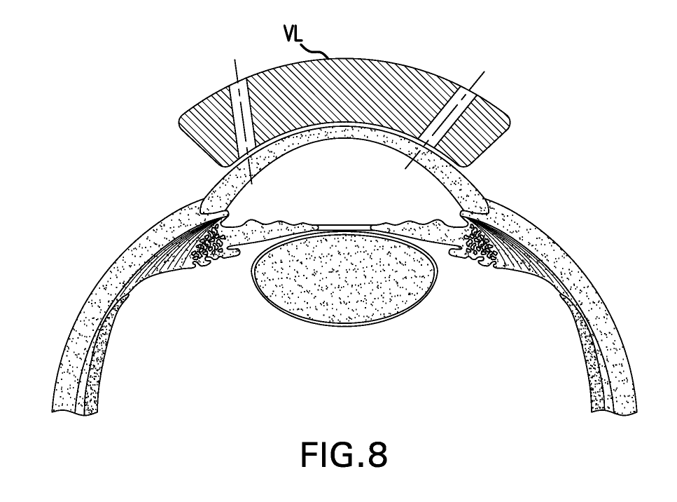

FIG. 8 shows a cross-sectional view of the eye. A viewing lens VL (such as a gonioscopy lens represented schematically in FIG. 8) is positioned adjacent the cornea. The viewing lens VL enables viewing of internal regions of the eye, such as the scleral spur and iris root junction, from a location outside the eye. A surgeon may use the viewing lens VL during delivery of the implant into the eye. The viewing lens VL may have a shape or cutout that permits the surgeon to use the viewing lens VL in a manner that does not cover or impede access to the corneal incision. It should also be appreciated that a viewing lens need not be used.

An endoscope may also be used during delivery to aid in visualization. For example, a twenty-one to twenty-five gauge endoscope may be coupled to the implant during delivery such as by mounting the endoscope along the side of the implant or by mounting the endoscope coaxially within the implant. Ultrasonic guidance may be used as well using high resolution bio-microscopy, optical coherence tomography, and the like. Alternatively, a small endoscope may be inserted though a second limbal incision in the eye to image the tissue during the procedure.

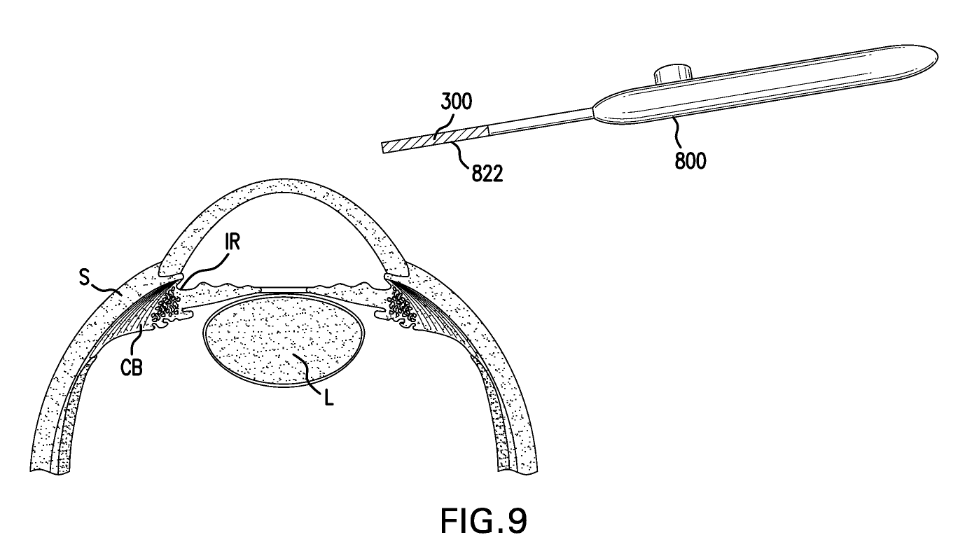

Referring to FIG. 9, in an initial step, at least one implant 300 may be mounted into a delivery system 800 for delivery into the eye. Implant 300 may be mounted into delivery system 800 such as by radially compressing and inserting implant 300 into the delivery tube 822 of instrument 800. The eye may be viewed through the viewing lens VL or other viewing means such as is described above, in order to ascertain the location where implant 300 is to be delivered. At least one goal is to deliver implant 300 in the eye so that it is positioned such that implant 300 provides a fluid pathway between the anterior chamber and the supraciliary space and/or the suprachoroidal space.

Delivery system 800 is positioned such that distal tip 823 of delivery tube 822 or implant 300 itself may be passed through a small corneal incision. In this regard, an incision may be made through the eye, such as near the limbus of the cornea. The incision may be very close to the limbus, such as either at the level of the limbus or within 2 mm of the limbus in the clear cornea. For example, a knife-tipped device or diamond knife may be used to make the incision to enter the cornea. As described above, the properties of delivery tube 822 such as material, material properties, dimensions, compliance, flexibility etc. contribute in part to the blunt dissection of the eye tissue and ensure that the implantation pathway substantially follows the boundary between tissue layers, for example between tissue layers such as the sclera S and ciliary body CB.

The corneal incision CI has a size that is sufficient to permit passage of the implant and delivery tube 822 therethrough. In one embodiment, the incision CI may be about 1 mm in size. In another embodiment, the incision CI may be no greater than about 2.85 mm in size. In another embodiment, the incision CI may be no greater than about 2.85 mm and may be greater than about 1.5 mm. Incisions may be made to be self-sealing incisions. For clarity of illustration, the drawing is not to scale and the viewing lens VL is not shown in FIG. 9.

After passing through the corneal incision, a delivery tube tip 823 may approach the iris root IR from the same side of the anterior chamber AC as the deployment location such that delivery tube 822 avoids being advanced across the iris. Alternately, delivery tube tip 823 may approach the insertion location from across the anterior chamber AC such that delivery tube tip 823 is advanced across the iris and or the anterior chamber toward the opposite iris root. Delivery tube 822 may approach the iris root IR along a variety of pathways. Delivery tube 822 may not necessarily cross over the eye and may not intersect the center axis of the eye. In other words, the corneal incision and the location where the implant is implanted at the iris root IR may be in the same quadrant (if the eye is viewed from the front and divided into four quadrants). Also, the pathway of the implant from the corneal incision to the iris root IR may not pass through the centerline of the eye to avoid touching or damaging the lens L.

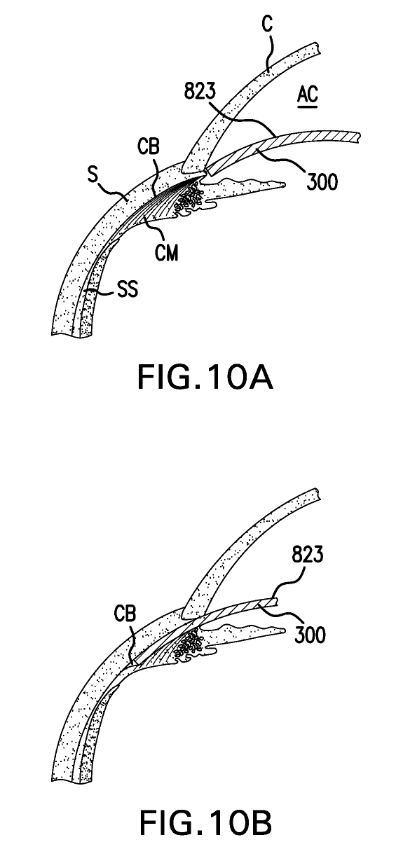

FIGS. 10A-10B show enlarged views of the anterior region of the eye. After insertion through the corneal incision, the implant mounted inside delivery tube 822 is advanced through the cornea into the anterior chamber along a pathway that enables the implant to be delivered to a position such that the implant provides a flow passageway from the anterior chamber into the supraciliary space and/or the suprachoroidal space. Delivery tube 822 travels along a pathway that is toward the scleral spur such that the delivery tube tip 823 passes near the scleral spur on the way to the supraciliary space and/or the suprachoroidal space. The scleral spur is an anatomic landmark on the wall of the angle of the eye. The scleral spur is above the level of the iris but below the level of the trabecular meshwork. In some eyes, the scleral spur can be masked by the lower band of the pigmented trabecular meshwork and be directly behind it. Delivery tube 822 may not pass through the scleral spur during delivery. Rather, delivery tube 822 may dissect the tissue boundary between the sclera and the ciliary body, entering near the iris root. Delivery tube 822 may penetrate the iris root or a region of the ciliary body or the iris root part of the ciliary body near its tissue border with the scleral spur. The combination of delivery tube properties and the angle of approach may allow the procedure to be performed “blind” as the instrument delivery tube tip 823 follows the inner curve of the scleral wall to dissect the tissue and create a channel in the tissue boundary to connect the anterior chamber to the supraciliary space and/or the suprachoroidal space. The surgeon may rotate or reposition the handle of delivery device 800 in order to obtain a proper approach trajectory for distal tip 823 of delivery tube 822. Delivery tube 822 may be pre-shaped, steerable, articulating, or shapeable in a manner that facilitates the applier approaching the iris root and the supraciliary space and/or the suprachoroidal space along a proper angle or pathway.

FIG. 10A shows distal tip 823 of delivery tube 822 positioned within the supraciliary space between the ciliary body CB and the sclera S. FIG. 10A shows the implant compressed and inserted inside applier tube 822. As delivery tube 822 advances through tissue, distal tip 823 causes the sclera S to dissect away or otherwise separate from the ciliary body CB, creating a small channel. A variety of parameters including the shape, material, material properties, diameter, flexibility, compliance, pre-curvature and tip shape of delivery tube 822 may make it more inclined to follow the tissue boundary between the ciliary body and the inner wall of the sclera. Delivery tube 822 containing the compressed implant is continuously advanced into the eye, until distal tip 823 is located at or near the end of the supraciliary space, near the beginning of the suprachoroidal space such that a first portion of the implant is positioned within the supraciliary space and is able to communicate fluid to the suprachoroidal space, and a second portion is positioned within the anterior chamber and is able to communicate fluid from the anterior chamber. In one embodiment, at least 1 mm to 2 mm of the implant (along the length) remains in the anterior chamber. The implant is then released from delivery tube 822 by retracting delivery tube 822 thereby ejecting the implant into the tissue channel created by the delivery tube advancement. The implant may expand in place when delivery tube 822 is retracted without moving greatly in the tissue or changing its length.

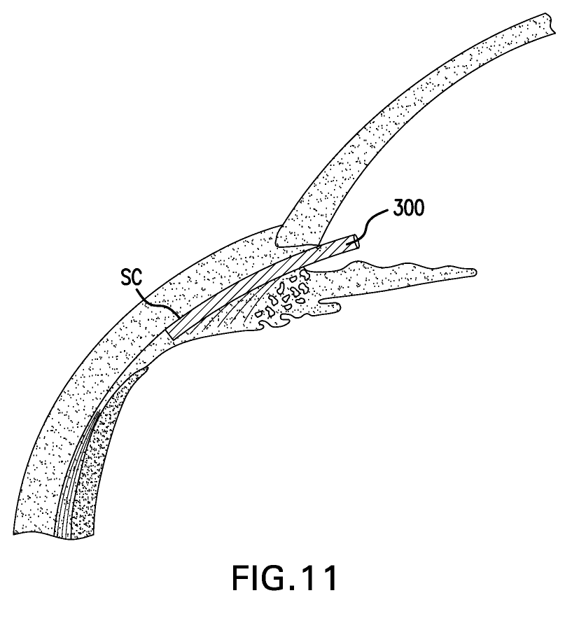

FIG. 11 shows the implant that has been placed into the eye by the retraction of delivery tube 822 while in the supraciliary space, allowing the implant to exit from delivery tube 822 and to expand in the supraciliary channel created by delivery tube 822 in the surgical procedure described in FIGS. 10A-10B.

Instrument Mechanisms

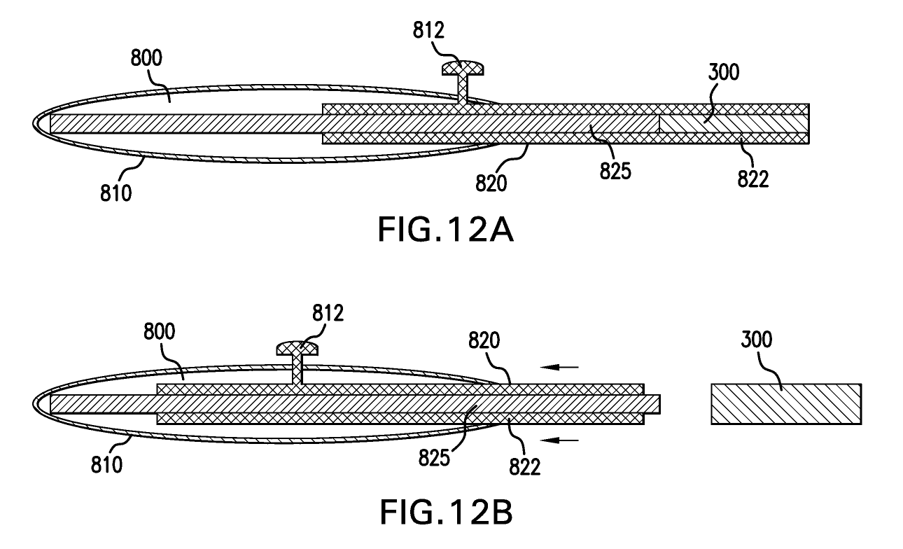

FIGS. 12A-12B shows a delivery instrument 800 that may be used to deliver the oblong porous implant into the eye both before and after releasing the implant from the delivery tube 822. Delivery instrument 800 may be used with implant 300, as illustrated, or any other suitable implant.

Delivery instrument 800 has a delivery tube 822 that may be loaded with a compressed implant which is inserted so that the proximal end rests against deployment stop 825. Delivery instrument 800 may be loaded with the compressed implant as shown in FIG. 12A. After the implant is compressed and loaded into delivery instrument 800, delivery instrument 800 containing the implant may be inserted into the eye to the location desired in the supraciliary space and/or the suprachoroidal space to release and expand the implant as shown in FIG. 12B. The release and expansion of the compressed implant may be achieved by retracting delivery tube 822 against the stop 825 by pulling back on a delivery tube retraction button 812, thereby causing the compressed implant to be ejected from delivery tube 822 and to expand in the tissue as it is expelled. The release and expansion of the implant is shown in FIG. 12B. Delivery tube 822 may include a mark or reflective band that indicates where the proximal end of the compressed implant is located inside of it to assist the surgeon to place delivery tube 822 in a location that will eject and expand the implant so that the proximal end of the implant is located in the desired location in the eye such as in the anterior chamber near the iris root, for example.

The loading of the implant into delivery tube 822 may be facilitated by loading tools that are able to radially compress the implant and push it into delivery tube 822 in a compressed shape. These loading tools may include split tube clamps that radially compress the implant when they are clamped over it, or elongate toroidal balloons that are inflated to radially compress the implant. A push rod may be used to push the compressed implant longitudinally to transfer the compressed implant from the loading tool into delivery tool 800. Pushing the implant through tapered tubes may also be used to compress the implant and transfer the implant into delivery tube 822. Non-toxic, biocompatible lubricants may be used to reduce the friction during the compression and loading of the implant into delivery tube 822. The use of heat shrink tubing to compress and transfer implant into delivery tube 822 may also be used, and the use of resorbable heat shrink tubing that may be left on the implant is a possible option, although this is not a requirement to load the implant using this method. The use of a collapsible braided tube structure may also be used to compress and load the implant into the delivery tube 822. To use a braid to load the implant, the implant must be able to slide past the inner braid fibers as it is being radially compressed by stretching the braid to a smaller diameter, and lubricants may be used to make this technique work better. Using braided structures that are coated with elastomeric polymers so as to make them a collapsible tube may also be used to compress and load the implant into delivery tube 822. Rolling the expanded implant to a smaller diameter is another technique that may be used to compress and load the implant into delivery tube 822. The rolled implant may be loaded quickly before the implant recovers and expands from this type of compression. Liquid agents may be used to delay the expansion after rolling to allow more time to load the compressed implant into delivery tube 822. Yet another method to compress implant and load it into delivery tube 822 would be to lay it inside a flexible tube and to pinch and pull the tubing wall along its length into a gap or slit between two long bars or rollers along its length to compress the implant in the loop of tubing left on one side of the rollers.

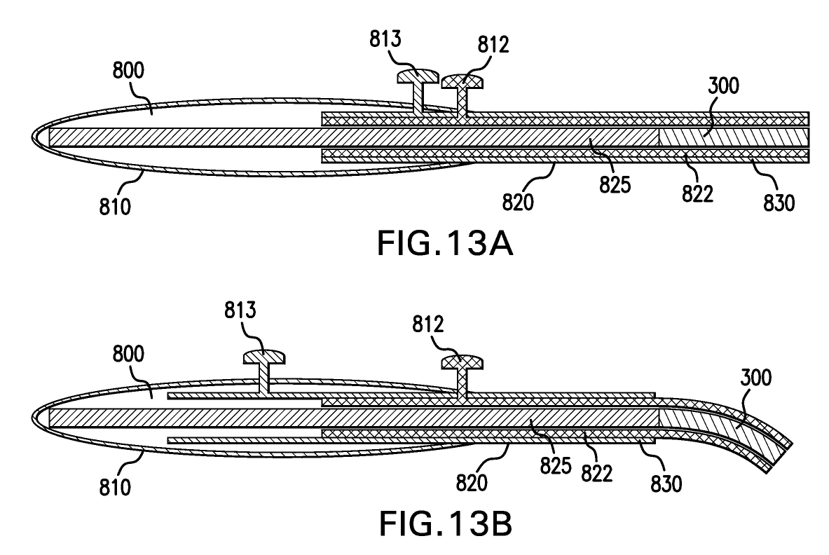

FIGS. 13A-13B show another embodiment of delivery instrument 800 that includes an additional straightening tube 830 that may be extended or retracted over delivery tube 822 so that distal tip 823 of delivery tube 822 will change from a straight to a curved shape. Straightening tube 830 may be retracted using button 813 thereby exposing distal tip 823 of delivery tube 822 which may have a memory shape that is curved and that it changes to when straightening tube 830 is retracted. The compressed implant inside curved delivery tube 822 may de delivered by retracting delivery tube 822 against stop 825, thereby ejecting the implant from delivery tube 822 allowing it to expand in place in the desired eye tissue location.

Implant Delivery System

There are now described devices and methods for delivering and deploying implant described herein into the eye. In an embodiment, a delivery system is used to deliver the implant into the eye such that the implant provides fluid communication between the anterior chamber and the supraciliary and or the suprachoroidal space.

FIGS. 12A-12B shows an exemplary delivery system 800 that may be used to deliver the implant into the eye. It should be appreciated that delivery system 800 in FIGS. 12A-12B is exemplary and that variations in the structure, shape and actuation of delivery system 800 are possible. Delivery instrument 800 may be used with implant 300, as illustrated, or any other suitable implant.

Delivery system 800 may include a handle component 810 that controls an implant placement mechanism, and a delivery component 820 that removably contains the compressed implant for delivery and expansion of the implant into the eye. Delivery component 820 may include an elongate delivery tube 822 that is sized and shaped to be inserted longitudinally around the implant. In one embodiment, the inner cross-sectional area of delivery tube 822 may be at least about 0.125 mm². In another embodiment, the inner cross-sectional area of delivery tube 822 may be at least about 2.0 mm². In one embodiment, delivery tube 822 may have a blunt distal tip 823, although it may also be sharp. Delivery tube 822 may have a cross-sectional shape that complements the cross-sectional shape of the implant and need not be circular. Delivery tube 822 may be straight or it can be curved along all or a portion of its length in order to facilitate proper placement through the cornea and into the supraciliary space. Delivery tube 822 may be more rigid than the implant such that it constrains the implant in a compressed, insertion configuration. Although delivery tube 822 may be more rigid than the implant, it may still remain flexible and compliant enough to allow for blunt dissection such as between the tissue layers of the sclera and choroid or the sclera and the ciliary body and able to follow the natural curve of the inner scleral wall. In one embodiment shown in FIGS. 13A-13B, delivery tube 822 may be made to change shape by covering it with a straightening tube 830, and then retracting tube 830 to allow delivery tube 822 to change from a straight to curved shape.

The outer diameter of delivery tube 822 can be selected based on the material and flexibility of the material used for delivery tube 822. A delivery tube 822 made of nitinol, for example, can have an outer diameter of about 0.5 mm. Nitinol is a superelastic metal that is quite bendable, yet is stiff enough to be pushed through the iris root and the ciliary body to reach and hug the curve of the inner scleral wall during blunt dissection along the boundary between the sclera and the tissues adjacent to the inner scleral wall. When combined with other features of delivery tube 822, for example a blunt tip 823, a nitinol delivery tube 822 having an outer diameter of about 0.5 mm may be used to gently dissect the tissue layers while avoiding tunneling or piercing one or both the inner scleral wall and choroid. Stainless steel spring wire is another material that may be used for delivery tube 822. Stainless steel is generally slightly stiffer than nitinol. Thus, the wall thickness of a delivery tube 822 made of stainless steel wire may need to be somewhat smaller than the wall thickness for a delivery tube made of nitinol in order to achieve the same performance during blunt dissection. In one embodiment, delivery tube 822 has an outer diameter of about 0.5 mm. It should be appreciated that for a given material's flexibility, the outer diameter of delivery tube 822 may be determined and extrapolated for a delivery tube 822 of a different material having a different degree of flexibility. Other materials which may be used for delivery tube 822 include compliant flexible tubes made from a polymer or reinforced polymer composite tubes made and reinforced using high-strength fibers, wires, or other fillers.

A variety of parameters including the shape, material, material properties, diameter, flexibility, compliance, pre-curvature and tip shape of delivery tube 822 may impact the performance of delivery tube 822 during gentle, blunt tissue dissection. These same parameters may be important for the compressed implant since it sits inside delivery tube 822 and may act to help the tissue dissection and to keep tissue from entering delivery tube 822 during the surgical procedure. It may be important that delivery tube 822 be able to penetrate certain tissues but to avoid the penetration of other tissues. For example, in one embodiment, it may be advantageous that delivery tube 822 be capable of penetrating the iris root or the ciliary body. The same delivery tube 822 may beneficially be incapable or have difficulty penetrating the inner wall of the sclera so that it can use the boundary of the inner scleral wall to repel penetration by delivery tube tip 823 and guide the dissection between the tissue boundaries adjacent to the inner wall of the sclera. It should also be appreciated that the column strength of the compressed implant may be sufficient to permit the implant to tunnel through certain eye tissues into the supraciliary space and/or the suprachoroidal space.

Manufacture of Shape Changing Implants

The dimensions of the implants described herein may vary. In one embodiment, the implant may have a length in the range of 3.0 mm to 30.0 mm, and an inner cross-sectional area for a flow path in the range of 0.1 mm² to 3.0 mm². In another embodiment, the inner cross-sectional area may be 0.125 mm², 0.28 mm², or 0.78 mm². In the event that multiple implants are used, and for example each implant is 0.125 mm², the fully implanted device may create a length of 3.0 mm to 30.0 mm, although the length may be outside this range. One embodiment of the implant is 7.5 mm long, and 0.28 mm² in cross sectional area. Another embodiment of the implant is 9.0 mm long.

The implants described herein including their shape changing portion(s) can be made of various biocompatible materials. In one embodiment, the implants may be manufactured of synthetic polymeric materials that are porous and that show reversible compression and may be deformed such that they return almost entirely to their “original” expanded porous shape when the compression is released. The reversible deformation of the implant, even at higher body temperatures, is a desirable characteristic.

The implant or portion(s) thereof may be made of various clean biocompatible materials, including, for example, synthetic polymers, block copolymers, thermoset polymers, synthetic rubbers including silicone polymers, thermoplastic polymers including thermoplastic elastomers, polyolefins, polyimides, polyesters, polyamides, polyaramids, polyethers, polyglycols, acrylic polymers including hydrogel versions, polyflouro polymers, polyurethanes, Nitinol, platinum, stainless steel, colbalt chrome alloys, molybdenum, titanium and its alloys, or any other suitable polymer, metal, metal alloy, or ceramic biocompatible material or combinations thereof. The material of manufacture is desirably selected to have material properties suited for the particular function of the implant or portion thereof.

Other materials of manufacture or materials with which the implant can be coated or manufactured entirely include silicone, thermoplastic elastomers (HYTREL, KRATON, PEBAX), certain polyolefin or polyolefin blends, elastomeric alloys, polyurethanes, thermoplastic copolyester, polyether block amides, polyamides (such as Nylon), block copolymer polyurethanes (such as LYCRA). Some other exemplary materials include fluoropolymer (such as FEP and PVDF), polyester, ePTFE (also known as GORETEX), FEP laminated into nodes of ePTFE, acrylic, low glass transition temperature acrylics, silver coatings (such as via a CVD process), gold, polypropylene, poly(methyl methacrylate) (PMMA), PolyEthylene Terephthalate (PET), Polyethylene (PE), PLLA, parylene, PEEK, polysulfone, polyamideimides (PAI) and liquid crystal polymers.

It should also be appreciated that almost all polymers may be made to be porous by incorporating air or void volumes into their bulk, for example, by using blowing agents or poregens. Methods to make porous structures include processes that generate pore structure inside of polymers as they are formed, such as foaming with gas, the use of pore forming or foam making agents (sometimes known as blowing agents), which can be either physical (example is gas injection or phase change of carbon dioxide) or chemical (example is use of bicarbonate to produce gas), processes that form pores by removing or etching away cast in microspheres using solvent, heat, or other methods, solid and hollow microsphere sintering, fluid bed and powder coating, electro spinning, etc.

In order to maintain a low profile, sputtering techniques can be employed to coat the implant.

Any of the embodiments of the implants described herein may be coated on the inner or outer surface with one or more drugs or other materials, wherein the drug or material maintains the patency of the lumen by preventing scarring, or encourages ingrowth of tissue to assist with retention of the implant within the eye or to prevent leakage around the implant. The drug may also be used for treatment of disease. The implant may be coated on its inner or outer surface with a therapeutic agent, such as a steroid, an antibiotic, an anti-inflammatory agent, an anti-coagulant, an anti-glaucomatous agent, an anti-proliferative, or any combination thereof. The drug or therapeutic agent may be applied in a number of ways. Also the drug may be embedded in another polymer so as to diffuse out to the surrounding areas around the implant or released from a resorbable polymer that is coated inside or outside or on both of these surfaces of the implant.

The shape change porous portion of the implant may be enhanced by one or more post-processing steps. Thermoplastic materials, including thermoplastic elastomers (TPEs), are characterized by labile cross-links that are reversible and can be broken when melted. This property of TPEs makes them easy to use from a manufacturing standpoint. The shape changing portion(s) of a porous thermoplastic implant may be further processed by imparting more permanent cross-links such as through heat, addition of ultra violet (UV) or free radical cross linking agents, multifunctional chemical cross linking agents or radiation such as electron beam exposure, gamma-radiation or UV light. Thermo sets and cross-linked polymers can also be used instead of, or in addition to thermoplastics that are later modified.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. An ocular implant, comprising
an elongate member having a flow pathway,
at least one inflow area communicating with the flow pathway, and
an outflow area communicating with the flow pathway,
wherein the elongate member includes at least one porous structure and is configured to transition between a compressed shape when compressed and an expanded shape when uncompressed;
wherein the elongate member is configured to be positioned in an eye such that the inflow area communicates with an anterior chamber of the eye and the outflow area communicates with a region of the eye that will increase aqueous outflow to help maintain a proper pressure of the eye.

2. An implant as in claim 1, wherein the elongate member is configured to transition from the compressed shape to the expanded shape without a substantial change in a length of the elongate member.

3. An implant as in claim 1, wherein at least one porous structure is permeable to a flow of aqueous and other body fluids.

4. An implant as in claim 1, wherein at least part of an outer surface of the elongate member includes pores having a pore size and pore size distribution that prevents excessive fibrosis and scarring of the elongate member.

5. An implant as in claim 4, wherein substantially all the pores have a similar size, wherein a mean size of the pores is between about 20 and about 60 micrometers, wherein substantially all the pores are each connected to adjacent pores, and wherein the pores allow permeable flow of fluids between the pores.

6. An implant as in claim 1, wherein at least part of an outer surface of the elongate member has a pore size and pore size distribution that allows fibrous in growth into the elongate member.

7. An implant as in claim 1, wherein at least part of an outer surface of the elongate member has a surface that helps prevent migration of the elongate member in tissue.

8. An implant in claim 1, wherein the elongate member is in a state of reduced diameter under compression and transitions to a state of enlarged diameter upon removal of compression.

9. An implant in claim 2, wherein the elongate member is in a state of reduced diameter under compression and transitions to a state of enlarged diameter upon removal of compression.

10-25. (canceled)

26. A method of implanting an ocular device into an eye, comprising:
forming an incision in a cornea of the eye;
compressing an implant;
inserting the implant through the incision into an anterior chamber of the eye;
passing the implant along a pathway from the anterior chamber into a supraciliary space and/or a suprachoroidal space of the eye;
positioning the implant in a first position such that a proximal portion of the implant communicates with the anterior chamber and a distal portion of the implant communicates with the supraciliary space and/or the suprachoroidal space to provide a path between the supraciliary space and/or the suprachoroidal space and the anterior chamber; and
permitting the implant to expand to an expanded shape, wherein the implant defines a fluid passageway in the expanded shape that allows fluid to communicate between the anterior chamber and the supraciliary space and/or the suprachoroidal space.

27. A method as in claim 26, wherein the implant is at least partially formed of a porous structure.

28. A method as in claim 26, wherein the implant includes a first portion formed at least partially of a porous structure and a second portion formed at least partially of a non-porous structure.

29. A method as in claim 26, further comprising inserting the implant into a delivery tube of a delivery device such that the delivery tube exerts a compressive force on the implant.

30. A method as in claim 26, comprising releasing the implant from compression after positioning the implant in the first position.

31. A method as in claim 26, wherein the fluid passageway comprises a lumen that extends through the implant.

32. A method as in claim 26, wherein the fluid passageway comprises a permeable porous channel that extends through the implant.

33. (canceled)

34. A method as in claim 26, wherein a distal end of the implant is compressed, and further comprising creating a dissection in a tissue boundary between a ciliary body and a sclera of the eye using the distal end of the implant.

35. A method as in claim 26, wherein passing the implant along a pathway from the anterior chamber into the supraciliary space comprises contacting a scleral spur of the eye and sliding the implant into the supraciliary space the just below the scleral spur.

36-56. (canceled)

57. An implant as in claim 1, wherein the region of the eye is a supraciliary space and/or a suprachoroidal space of the eye.

58. An implant as in claim 1, wherein the elongate member further comprises at least one non-porous structure.
